(19) United States
(12) Patent Application Publication
(10) Pub. No.: US 2025/0213300 A1
(43) Pub. Date: Jul. 3, 2025

(54) ELECTROSURGICAL RETURN ELECTRODE PLUG

(71) Applicant: Cilag GmbH International, Zug (CH)

(72) Inventors: Barry Worrell, Maineville, OH (US); Amy Krumm, Madeira, OH (US); John Minuth, Cottonwood Height, UT (US); Patrick Minnelli, Harrison, OH (US)

(21) Appl. No.: 18/398,534

(22) Filed: Dec. 28, 2023

(51) Int. Cl. A61B 18/16; H01B 5/00

(52) U.S. Cl. CPC A61B 18/16; H01B 5/00

(57) ABSTRACT

An electrosurgical return electrode includes a conductive layer and a plug connected thereto. The conductive layer is configured to conduct electrical current and has one or more apertures extending therethrough. The plug includes a base having one or more bosses configured to extend through the one or more apertures in the conductive layer. The plug also includes a cap disposed adjacent to the conductive layer and opposite to the base. The cap and the base are laser or ultrasonically welded together or the conductive layer, the base, and the cap are joined together via an adhesive.

### BACKGROUND

### 1. Technical Field

The present disclosure relates generally to electrosurgical return electrodes. More particularly, the present disclosure relates to electrical plugs or connections for electrosurgical return electrodes.

### 2. The Relevant Technology

In the area of electrosurgery, medical procedures of cutting tissue and/or cauterizing leaking blood vessels are performed by utilizing radio frequency (RF) electrical energy. As is known to those skilled in the medical arts, electrosurgery is widely used and offers many advantages including that of the use of a single surgical tool for both cutting and coagulation. The RF energy is produced by a wave generator or Electro-Surgical Unit (ESU) and transmitted to a patient's tissue through a hand-held electrode that is operated by a surgeon.

Monopolar electrosurgical generator systems have an active electrode that is applied by the surgeon to the patient at the surgical site to perform surgery and a return electrode that provides a return path from the patient back to the ESU.

Since the inception of electrosurgery, various types of return electrodes have been used. Initially, return electrodes consisted of flat stainless steel plates (which in later years were coated with a conductive gel) that were placed under the patient's buttocks, thigh, shoulders, or any location where gravity could ensure adequate contact area. In an effort to improve the safety of return electrodes, the flat steel plates were eventually replaced with flexible return electrodes. Like the steel plate electrodes, the flexible return electrodes are also coated with a conductive or dielectric polymer. Additionally, the flexible return electrodes have an adhesive border on them so they can be attached to the patient without the aid of gravity. These flexible return electrodes are commonly referred to as “sticky pads.”

One of the biggest improvements to electrosurgery came in the form of self-limiting return electrodes. Unlike sticky pads and steel plate return electrodes, self-limiting return electrodes are relatively large, thereby eliminating the need for conductive gels that may irritate a patient's skin. Additionally, self-limiting return electrodes typically employ geometries and materials whose impedance characteristics, at typically used electrosurgical frequencies, are such that the return electrode self-limits current densities (and corresponding temperature rises) to safe thresholds, should the contact area between the patient and the electrode be reduced below otherwise desirable levels. Furthermore, self-limiting return electrodes were specifically designed to evenly distribute the current density over the entire contact area between the patient and the return electrode in order to reduce the risk of patient burns.

Megadyne Medical Products and OKL Aukland have provided self-limiting return electrodes. Examples of Megadyne's return electrodes are described in U.S. Pat. Nos. 6,544,258, 7,166,102, and 10,085,791. OKL Aukland's return electrode is described in Chinese Patent Publication No. 201088627. These return electrodes include a conductive layer and outer dielectric layers disposed on opposing sides of the conductive layer. The return electrodes also include an electrical plug that can be used to electrically connect a cable to the conductive layer. The opposing end of the cable can be connected to the ESU.

The construction of the plugs on the Megadyne and OKL Aukland return electrodes present various challenges and drawbacks. The plugs are constructed using a multiple step overmolding process that is expensive and produces high levels of scrap material/waste.

An example of the plugs used on the Megadyne and OKL Aukland return electrodes is shown in FIGS. 1A-1C. As can be seen in FIG. 1A, the return electrode includes a conductive layer 100. As also shown in FIG. 1A, an electrical plug of the return electrode includes an interior structural component 102 that is formed of an acrylonitrile butadiene styrene (“ABS”) plastic during a first shot molding process. The ABS plastic extends over the front and back surfaces of the conductive layer 102, thereby securing the interior structural component 102 to the conductive layer 100. As shown in FIG. 1B, an outer layer 104 formed of a thermoplastic polyurethane (“TPU”) material is then overmolded onto the interior structural component 102 during a second shot process. The TPU material extends over all or substantially all of the ABS plastic, including on the front and back of the conductive layer 100. Thereafter, as shown in FIG. 1C, the dielectric layers 106 are attached to opposing sides of the conductive layer 100 and around the molded plug component.

The construction of the return electrodes with the overmolded plugs often cannot be completed by a single manufacturing facility due to the multiple specialized steps that are required. Accordingly, the return electrode (or components thereof (including the conductive layer 100)) has to be shipped to multiple different manufacturing facilities to have the plug molded onto the conductive layer 100 and then to have the dielectric layers 106 added. Shipping the return electrode to different manufacturing facilities for construction complicates the supply chain and manufacturing process, which results in higher production costs.

Thus, although various advances have been made in the electrosurgical arts, there remains room for improvement. For instance, electrical plugs for return electrodes that are simpler to manufacture and assemble are desirable.

### BRIEF SUMMARY

In accordance with some embodiments, an electrosurgical return electrode includes a conductive layer and a plug connected thereto. The conductive layer is configured to conduct electrical current and has one or more apertures extending therethrough. The plug includes a base having one or more bosses configured to extend through the one or more apertures in the conductive layer. The plug also includes a cap disposed adjacent to the conductive layer and opposite to the base. The cap and the base are laser or ultrasonically welded together or the conductive layer, the base, and the cap are joined together via an adhesive.

In accordance with some embodiments, an electrosurgical return electrode includes a conductive layer configured to conduct electrical current and having one or more apertures extending therethrough. The electrosurgical return electrode also includes a plug connected to the conductive layer. The plug includes a base having a structural component and an outer layer covering most of the structural component. The structural component includes one or more bosses that extend through the one or more apertures in the conductive layer. The one or more bosses are formed of a material that is responsive to a laser. The plug also includes a cap disposed adjacent to the conductive layer and opposite to the base. The cap has one or more recesses for receiving the one or more bosses therein. The cap is formed of a transparent or at least partially translucent material configured to allow for a laser to pass therethrough and into the one or more bosses.

In accordance with some embodiments, an electrosurgical return electrode includes a conductive layer configured to conduct electrical current and having one or more apertures extending therethrough. The return electrode also includes a plug connected to the conductive layer. The plug includes a base having a structural component and an outer layer covering at least a portion of the structural component. The structural component includes one or more bosses that are configured to extend through the one or more apertures in the conductive layer. The plug also includes a cap disposed adjacent to the conductive layer and opposite to the base. The cap has one or more recesses for receiving the one or more bosses therein. One or both of the base and the cap have one or more apertures extending therethough and that configured to have an adhesive injected therethrough to file voids between the conductive layer, the base, and the cap to secure the conductive layer, the base, and the cap together.

Additional features and advantages of the disclosed embodiments will be set forth in the description which follows, and in part will be obvious from the description, or may be learned by the practice of the disclosure. These and other features will become more fully apparent from the following description and appended claims, or may be learned by the practice of the present disclosure.

### BRIEF DESCRIPTION OF THE DRAWINGS

To further clarify the above and other advantages and features of the present disclosure, a more particular description will be rendered by reference to specific embodiments thereof which are illustrated in the appended drawings. It is appreciated that these drawings depict only illustrated embodiments of the disclosure and are therefore not to be considered limiting of its scope. The disclosure will be described and explained with additional specificity and detail through the use of the accompanying drawings in which:

FIGS. 1A-1C illustrate an example of previous return electrodes with an overmolded plug.

FIG. 2 is a perspective view of an electrosurgical return electrode according to one embodiment of the present disclosure;

FIG. 3 is a partial exploded view of the electrosurgical return electrode of FIG. 2;

FIGS. 4 and 5 are perspective views of a structural component of a plug of the electrosurgical return electrode of FIG. 2;

FIG. 6-8 are perspective views of base of the plug of the electrosurgical return electrode of FIG. 2;

FIGS. 9 and 10 are perspective views of a cap of the plug of the electrosurgical return electrode of FIG. 2; and

FIGS. 11 and 12 illustrate example steps for assembling the electrosurgical return electrode of FIG. 2.

FIG. 13 illustrates an exploded view a plug of an electrosurgical return electrode according to another example embodiment.

FIG. 14 illustrates a cross-sectional view of the plug of FIG. 13.

### DETAILED DESCRIPTION

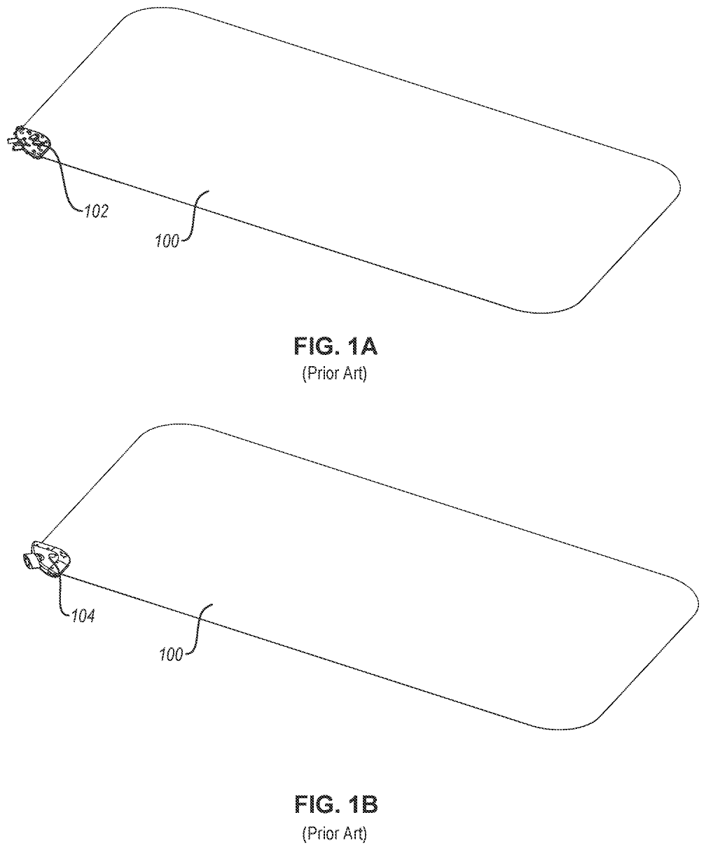

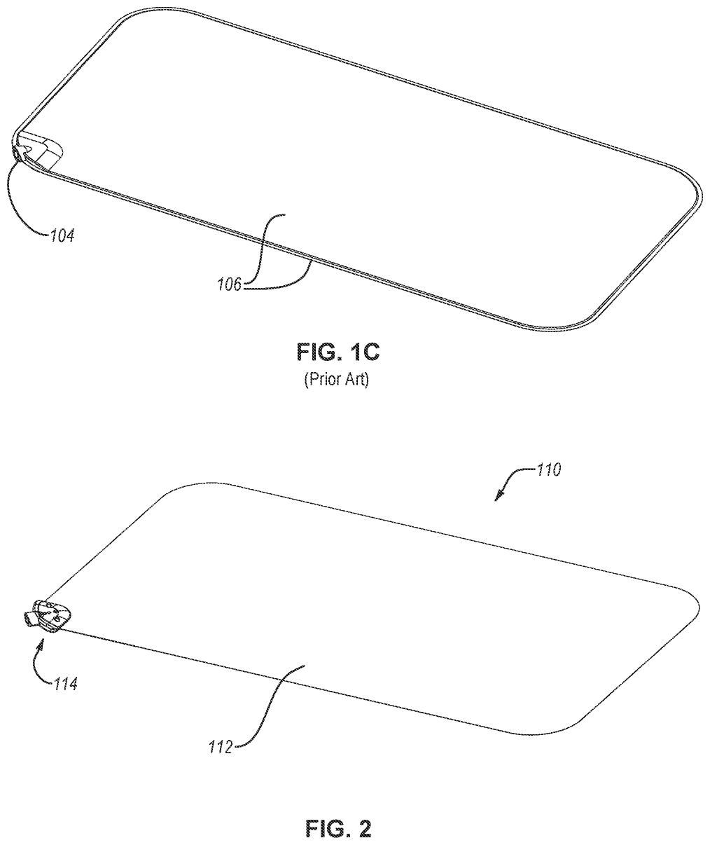

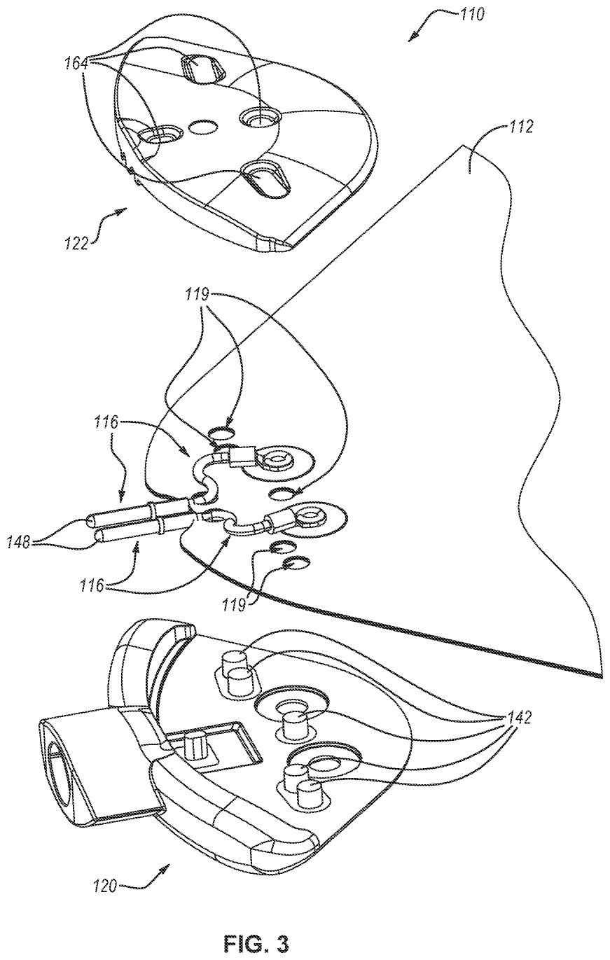

The present disclosure relates generally to electrosurgical return electrodes. More particularly, the present disclosure relates to electrical plugs or connections for electrosurgical return electrodes. FIGS. 2 and 3 illustrate a portion of an electrosurgical return electrode 110 according to the present disclosure. The portion of the electrosurgical return electrode 110 shown in FIGS. 2 and 3 includes a conductive layer 112 and a plug 114. Although not shown, the electrosurgical return electrode 110 may also include a dielectric layer on one or both sides thereof, similar to the dielectric layers 106 from FIG. 1C.

Although the electrosurgical return electrode 110 may include elements (e.g., conductive layer, plug, dielectric layers) that are generally similar to those of the return electrode of FIGS. 1A-1C, the features and construction of at least some of the components of the electrosurgical return electrode 110 are markedly different from the previous return electrodes. For instance, the construction of the plug 114 and the manner in which the plug 114 is secured to the conductive layer 112 is clearly different from the plug of the prior return electrodes.

FIG. 3 illustrates a partially exploded view of the plug 114 and the corner of the conductive layer 112 to which the plug 114 can be secured. As can be seen, electrical terminals 116 are connected to the conductive layer 112 so as to make an electrical connection therebetween. Ends of the electrical terminals 116 are configured to interface with an electrical cable to electrically connect the electrosurgical return electrode 110 to the ESU. The conductive layer 112 also includes a plurality of apertures 119 extending therethrough.

In the embodiment illustrated in FIG. 3, the plug 114 is formed of a base 120 (may also be referred to as a first half or first component) and a cap 122 (may also be referred to as a second half or second component). As described in more detail below, the base 120 and the cap 122 may be secured together with a portion of the conductive layer 112 disposed therebetween. The base 120 and the cap 122 may be secured to one another and/or to the conductive layer 112 so as to prevent unintended separation.

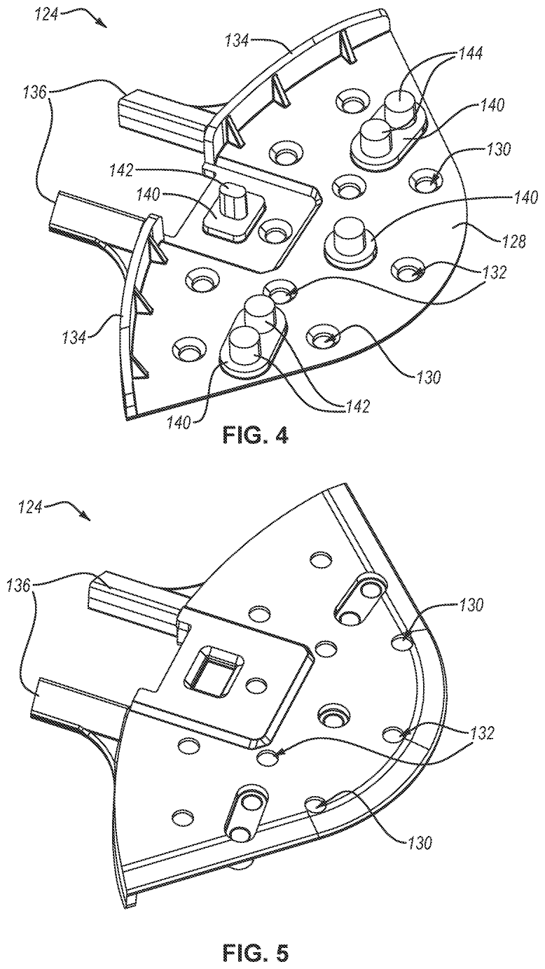

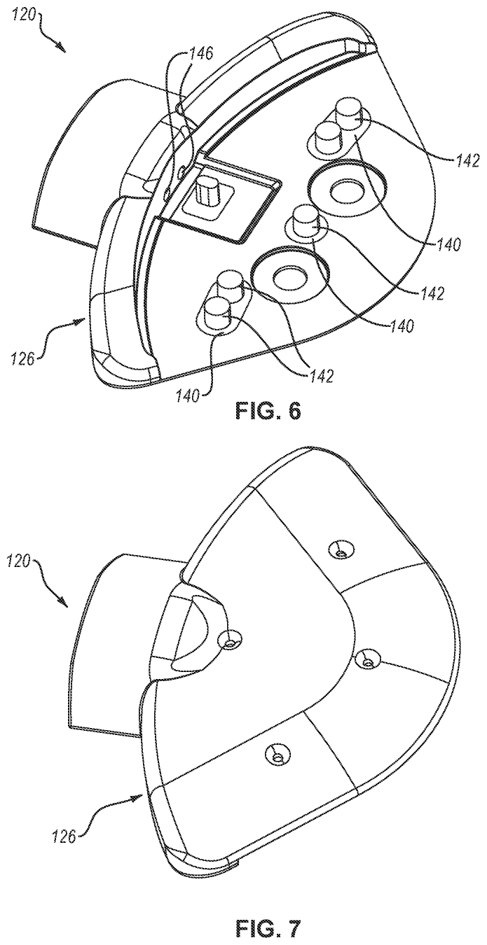

Attention is now directed to FIGS. 4-8, which illustrate the base 120 is greater detail and separate from the rest of the electrosurgical return electrode 110. In general, the base 120 includes a structural component 124 and an outer layer 126. In some embodiments, the base 120 is formed via a two shot injection molding process. For instance, the structural component 124 may be formed during a first shot injection molding process and then the outer layer 126 may be injection molded over the structural component during a second shot.

FIGS. 4 and 5 illustrate perspective views of opposing sides of the structural component 124. The structural component 124 may be formed of various generally rigid materials, such as a polyetherketone. In some embodiments, the structural component is formed of a dark colored material to facilitate laser welding to the cap 122, as described elsewhere herein. As noted, the structural component 124 may be formed via a molding process, such as injection molding. However, the structural component 124 may be formed via other processes as well.

The structural component 124 includes a platform 128. The platform 128 may include one or more apertures 130 therethrough and/or one or more apertures 132 therethrough. The aperture(s) s 130 may be configured to receive portions of the electrical terminals 116 (e.g., grommets, etc.) therein (see FIG. 3). The aperture(s) 132 may be configured to facilitate a secure connection between the structural component 124 and the outer layer 126. For instance, when the outer layer 126 is being molded on the structural component, the material forming the outer layer 126 may extend over opposing sides of the platform 128 can pass through the aperture(s) 132, thereby securing the outer layer 126 to the structural component 124.

The base 120 may also include a ridge 134 extending along a portion thereof. The ridge 134 may be configured to provide additional strength to the base 120. Additionally, the base 120 may include legs 136, 138 extending from the ridge 134 opposite to the platform 128.

The base 120 also includes a plurality of raised platforms 140 extending up from the platform 128. Extending up from the raised platforms 140 are a plurality of bosses 142 that are configured to facilitate connection between the base 120 and the cap 122, as discussed below. Each of the bosses 142 includes a top surface 144. In some embodiments, the top surfaces 144 lie within a single common plain.

FIGS. 6-8 illustrate perspective views of the base 120 from opposing sides and an end thereof. In the illustrated embodiment, the outer layer 126 has been added over the structural component 124. As noted, the outer layer 126 may be injection molded over the structural component 124. The outer layer 126 may be formed of a material that provides less structural rigidity or strength than the structural component 124. For instance, the outer layer 126 may be formed of a softer material having an Ra value of about 85, such as a thermoplastic polyurethane (“TPU”). The outer layer 126 may cover most of the structural component 124. However, the outer layer 126 may not cover the top surfaces of the raised platforms 140 or the bosses 142.

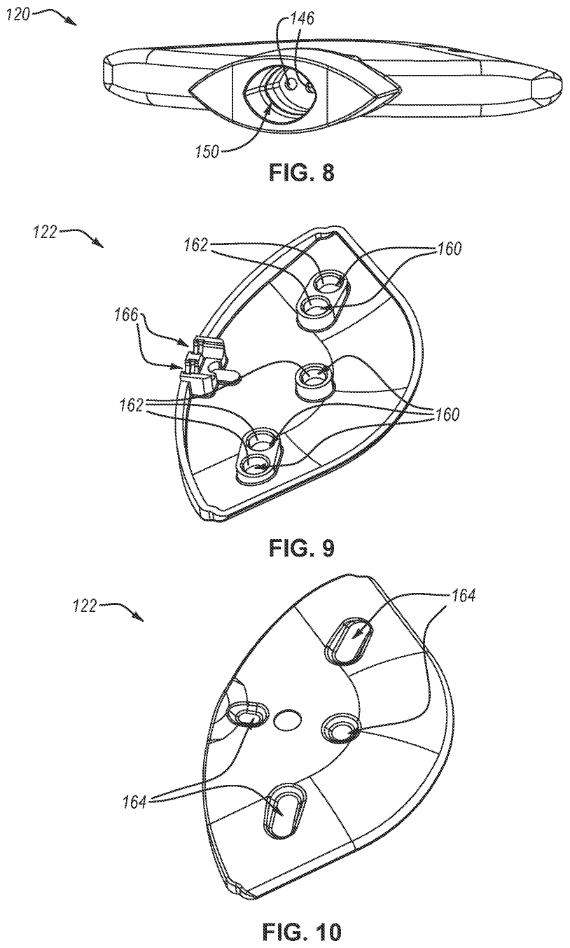

As can be seen in FIGS. 6 and 8, the outer layer 126 may form or have extending therethrough channels 146. The channels 146 may be sized and configured to have pins 148 of the electrical terminals 116 pass therethrough. The size of the channels 146 and the material used to form the outer layer 126 may cooperate to hold the pins 148 in a desired position and orientation. Additionally, the interface between the channels 146 and pins 148 may create a barrier to the ingress into the plug 114 of fluids or other contaminants.

As best seen in FIG. 8, the outer layer 126 may form a socket 150. The socket 150 may be sized and configured to receive a cable connector (from an ESU cable) at least partially therein. The pins 148 may extend through the channels 146 and into socket 150 to enable connection with the cable connector. The legs 136 of the structural component 124 shown in FIGS. 4 and 5 may extend on opposing sides of the socket 150 and may be configured to maintain the dimensional integrity of the socket 150 (e.g., to limit or prevent the portion of the outer layer 126 forming the socket 150 from shrinking or becoming deformed).

Attention is now directed to FIGS. 9 and 10, which illustrate the cap 122 in more detail and separate from the rest of the electrosurgical return electrode 110. FIG. 9 illustrates a side (interior side) of the cap 122 that faces the conductive layer 112 and the base 120, and FIG. 10 illustrates a side (exterior) of the cap 122 that faces away from the conductive layer 112 and the base 120.

As can be seen in FIG. 9, the interior side of the cap 122 includes a plurality of recesses 160. The recesses are arranged on the cap 122 in an arrangement that corresponds to the arrangement of the bosses 142 on the base 120. Additionally, the recesses 160 have outer dimensions that are at least as large as the outer dimensions of the bosses 142. Accordingly, the recesses 160 are configured have the bosses 142 inserted at least partially therein.

The recesses 160 can have bottom surfaces 162. The bottom surfaces 162 may all lie within a single common plain. Thus, when the bosses 142 are inserted into the recesses 160, the top surfaces 144 of the bosses 142 may engage with the bottom surfaces 162 of the recesses 160.

The thickness of the cap 122 at the bottom surfaces 162 may be relative thin. For instance, the thickness may be about 0.030 inches or about 0.040 inches thick. In order to achieve this thickness, the exterior of the cap 122 may have recesses 164 formed therein to reduce the thickness of the cap 122 in the desired areas.

The cap may be formed of a transparent or at least partially translucent material to allow for a laser to pass therethrough. The thickness of the cap 122 at the bottom surfaces 162 and the transparent or partially translucent material used to form the cap 122 may enable the cap 122 and the base 120 to be laser welded together, as described herein.

The interior of the cap 122 also includes retention features 166 configured to help hold the pins 148 of the electrical terminals 116 is a desired position and orientation. As shown in FIG. 9, the retention features 166 may include channels, grooves, or other contoured features that correspond in shape and size to the pins. The retention features 166 may also include one or more locating features (e.g., annular recess) that cooperate with one or more locating features (e.g., annular ring) on the pins 148 to help properly locate the pins 148.

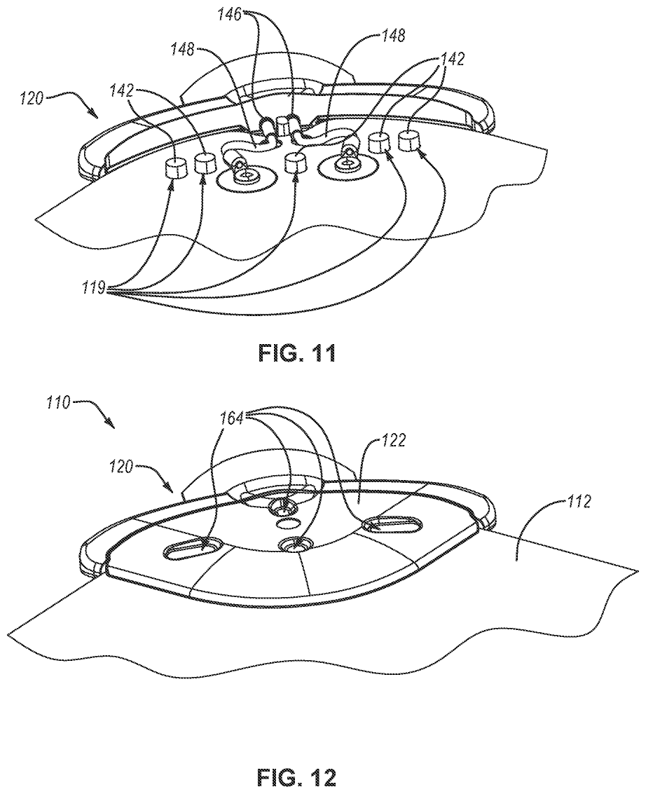

Attention is now directed to FIGS. 11 and 12, which illustrate some example steps for assembling the electrosurgical return electrode 110. Once the individual components (e.g., conductive layer 112, base 120, and cap 122) of the electrosurgical return electrode 110 have been manufactured, they can be assembled into a working unit.

As shown in FIG. 11, for instance, the assembly can include associating the base 120 with the conductive layer 112. This can include positioning the base 120 so that the bosses 142 extend through the apertures 119 in the conductive layer 112. This step can also include inserting the distal ends of the pins 148 into the channels 146 to a predetermined depth (e.g., so they extend into the socket 150 a desired distance). Additionally, the proximal ends of the pins 148 can be positioned on one of the raised platforms 140 to support the proximal ends thereof.

Next, the cap 122 can be positioned onto the conductive layer 112 and the base 120, as shown in FIG. 12. In the illustrated embodiment, there are two aspects to properly positioning the cap. First, the cap 122 is positioned so that the recesses 160 receive the bosses 142 therein so that the top surfaces of the bosses 142 engage the bottom surfaces 162 of the recesses 160. Second, the retention features 166 are aligned with and placed over the pins 148 (e.g., to the annular groove receives the annular rings on the pins 148).

Once the cap 122 is so positioned, the base 120 and the cap 122 can be secured together. For instance, a laser can be directed through the cap 122 at the recesses 160, 164. Because the cap 122 is formed of a transparent or at least partially translucent material, the laser can pass through the cap 122 and onto the top surfaces 144 of the bosses 142. The laser energy can cause the top surfaces 144 of the bosses 142 to melt and become bonded with the bottom surfaces 162 of the recesses 160 in the cap 122. In this manner, the base 120 and the cap 122 can be laser welded together. Once the base 120 and the cap 122 are secured together, the conductive layer 112 is secured therebetween due to the bosses 142 extending through the apertures 119 in the conductive layer 112.

Although the base 120 and the cap 122 are described herein as being laser welded together, this is merely exemplary. In other embodiments, the base and the cap can be ultrasonically welded together. The assembly process for the electrosurgical return electrode 110 would be similar to that described above. However, rather than passing a laser through the cap, ultrasonic vibration could be passed through the cap and into the bosses.

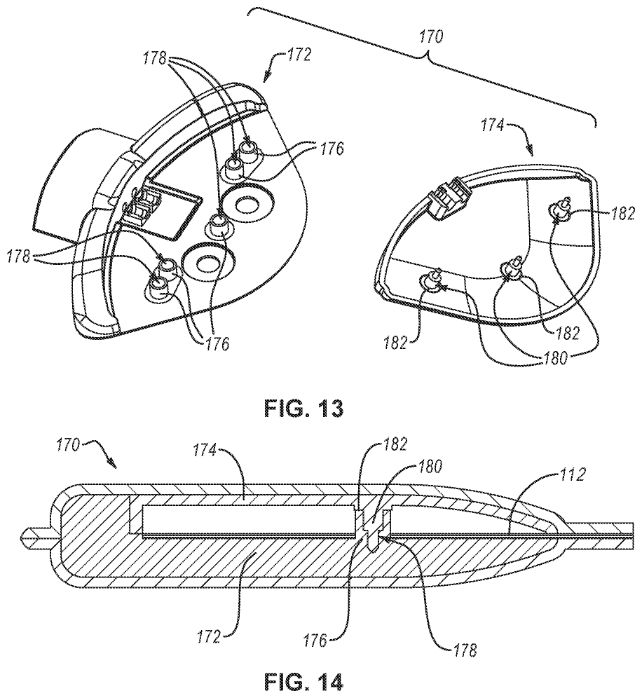

FIGS. 13 and 14 illustrate an example embodiment of a plug 170 (or components thereof) that is designed for ultrasonic welding. In the illustrated embodiment, the plug 170 includes a base 172 and a cap 174. The base 172 and the cap 174 may be similar or identical to the base 120 and the cap 122 in many respects. However, the base 172 and the cap 174 may have features that are designed to facilitate ultrasonic bonding therebetween.

For instance, an interior surface of the base 172 (as shown in FIG. 13) may include one or more bosses 176 having a recess 178 extending into an end thereof. Correspondingly, an interior surface of the cap 174 (as shown in FIG. 13) may include one or more posts 180 extending therefrom. The posts 180 may be arranged, sized, and shaped, so as to be received within the recess(es) 178 of the boss(es) 176.

In some embodiments, such as that shown in FIGS. 13 and 14, the recess(es) 178 and post(s) 180 may have stepped or tapered configurations. For instance, as shown, each of the posts 180 has a wider base portion and a narrower tip portion. Similarly, each of the recesses 178 includes a wider upper region and a narrower lower region configured to receive the wider base portion and the narrower tip portion, respectively, of the post 180. The narrower portions of the posts 180 may be configured to absorb the ultrasonic vibration, melt, and bond with the interior surfaces of the recesses 178. The narrower section serves to increase the energy density to facilitate melting of the plastic and the resultant welding.

For instance, as shown in FIG. 14, the plug 170 may be assembled with the conductive layer 112 therebetween. In the assembled configuration, the post(s) 180 extend into the recess(es) 178 of the boss(es) 176. As shown, the post(s) 180 may include a shoulder 182 that is configured to engage a top end of the boss(es) 176. The top ends of the bosses 176 may lie in a single, common plane. Similarly, the shoulders 182 of the posts 180 may lie in a single, common plane. As a result, the shoulders 182 and the tops of the bosses 176 may engage one another and ensure proper alignment of the base 172 and the cap 174.

With the plug 170 so assembled, ultrasonic vibrations can be applied to the base 172 and/or the cap 174 to cause the post(s) 180 (or tips thereof) to melt and bond with the interior of the recess(es) 178. In this manner, the base 172 and the cap 174 can be secured together with the conductive layer 112 therebetween.

In still other embodiments, the base and the cap may include one or more apertures extending therethrough and configured to receive an epoxy or other adhesive. In such an embodiment, the base and the cap can be held together as shown in FIGS. 12 and 14. The epoxy or other adhesive may then be injected through the apertures and into the spaces between the base, the cap, and the conductive layer 112. Once the epoxy or other adhesive is cured, the base, the cap, and the conductive layer 112 would be bonded together.

In still other embodiments, mating mechanical connections on the base and the cap may be used to secure the base and the cap together. For instance, the bosses and posts may include mating mechanical connection features, such as interlocking, friction fitting, or snap connections. In other embodiments, the base and the cap may have such mating mechanical connection features disposed around at least part of a perimeter thereof or in other areas thereof.

While the electrosurgical return electrode 110 is described herein as including electrical terminals 116 and a socket 150 that allow for selective connection to a separate ESU cable, this is merely exemplary. In other embodiments, the ESU cable may be integrated/hardwired into the electrosurgical return electrode. In such an embodiment, the electrical conductors in the ESU cable would extend through the plug and be connected to the conductive layer.

Moreover, although electrosurgical return electrode and plug components shown in the Figures as having particular shapes and sizes, it will be appreciated that electrosurgical return electrodes and plugs thereof according to the present disclosure could have a variety of other shapes and sizes.

The terms “approximately,” “about,” and “substantially” as used herein represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of a stated amount.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An electrosurgical return electrode comprising:
a conductive layer configured to conduct electrical current, the conductive layer having one or more apertures extending therethrough; and
a plug connected to the conductive layer, the plug comprising:
a base having one or more bosses configured to extend through the one or more apertures in the conductive layer; and
a cap disposed adjacent to the conductive layer and opposite to the base, the cap and the base being laser or ultrasonically welded together or the conductive layer, the base, and the cap being joined together via an adhesive.

2. The electrosurgical return electrode according to claim 1, wherein each of the one or more bosses comprise a recess in an end thereof, and the cap comprises one or more posts on an interior surface that configured to be received in the recesses of the one or more bosses.

3. The electrosurgical return electrode according to claim 2, wherein the one or more bosses comprise a plurality of bosses with top ends that lie within a single common plain.

4. The electrosurgical return electrode according to claim 3, wherein the one or more posts comprise a plurality of posts, each of the posts having a shoulder that lies within a single common plain.

5. The electrosurgical return electrode according to claim 2, wherein the one or more posts comprise a stepped or tapered configuration.

6. The electrosurgical return electrode according to claim 5, wherein the recesses in the one or more bosses comprise a stepped or tapered configuration on an interior surface thereof.

7. The electrosurgical return electrode according to claim 1, wherein the base comprises an internal structural component and an outer layer formed of a softer material than the structural component.

8. The electrosurgical return electrode according to claim 7, wherein the structural component comprises a platform from which the one or more bosses extend.

9. The electrosurgical return electrode according to claim 8, wherein the platform comprises one or more apertures extending therethough, the one or more apertures having part of the outer layer passing therethrough.

10. The electrosurgical return electrode according to claim 9, wherein the one or more bosses are not covered by the outer layer.

11. The electrosurgical return electrode according to claim 7, wherein the outer layer comprises a socket and one or more channels leading to the socket.

12. The electrosurgical return electrode according to claim 1, wherein the base and the cap have one or more structural features that support one or more electrical terminals connected to the conductive layer.

13. An electrosurgical return electrode comprising:
a conductive layer configured to conduct electrical current, the conductive layer having one or more apertures extending therethrough; and
a plug connected to the conductive layer, the plug comprising:
a base having a structural component and an outer layer covering most of the structural component, the structural component comprising one or more bosses that extend through the one or more apertures in the conductive layer, the one or more bosses being formed of a material that is responsive to a laser; and
a cap disposed adjacent to the conductive layer and opposite to the base, the cap having one or more recesses for receiving the one or more bosses therein, the cap being formed of a transparent or at least partially translucent material configured to allow for a laser to pass therethrough and into the one or more bosses.

14. The electrosurgical return electrode according to claim 13, wherein the cap has a thickness of about 0.030 inches or about 0.040 inches adjacent to the one or more recesses.

15. The electrosurgical return electrode according to claim 13, further comprising one or more electrical terminals connected to the conductive layer.

16. The electrosurgical return electrode according to claim 15, wherein the outer layer comprises one or more channels therein, the one or more channels being configured to receive the one or more electrical terminals therein and prevent fluid from passing therebetween.

17. The electrosurgical return electrode according to claim 16, wherein the outer layer comprises a socket configured to receive a cable connector for selective connection with the one or more electrical terminals.

18. The electrosurgical return electrode according to claim 17, wherein the structural component comprises one or more legs that extends through the outer layer and adjacent to the socket.

19. An electrosurgical return electrode comprising:
a conductive layer configured to conduct electrical current, the conductive layer having one or more apertures extending therethrough; and
a plug connected to the conductive layer, the plug comprising:
a base having a structural component and an outer layer covering at least a portion of the structural component, the structural component comprising one or more bosses that are configured to extend through the one or more apertures in the conductive layer; and
a cap disposed adjacent to the conductive layer and opposite to the base, the cap having one or more features configured to engage with the one or more bosses,
one or both of the base and the cap having one or more apertures extending therethough and configured to have an adhesive injected therethrough to file voids between the conductive layer, the base, and the cap to secure the conductive layer, the base, and the cap together.

20. The electrosurgical return electrode according to 19, further comprising a connector cable hardwired to the conductive layer, the connector cable comprising one or more conductors that extends through the plug.
